(19) United States
(12) Patent Application Publication
(10) Pub. No.: US 2008/0097266 A1
(43) Pub. Date: Apr. 24, 2008

(54) Patient protection device and method

(76) Inventor: Gina Marie Santosuosso, Boca Raton, FL (US)

Correspondence Address: ROBERT M. DOWNEY, P.A., 6751 N. FEDERAL HWY., SUITE 300, BOCA RATON, FL, 33487, US

(21) Appl. No.: 11/506,033

(22) Filed: Aug. 17, 2006

(51) Int. Cl. A61F 5/00; G09B 23/28

(52) U.S. Cl. 602/3; 434/262

(57) ABSTRACT

A device and method for alerting attending medical personnel of a patient's potential risk of injury due to unwanted blood pressure measurements, intravenous access and venipuncture includes placing a sleeve on the patient's affected arm or leg. The sleeve is formed of a latex free soft fabric which is stretchable to provide a snug, comfortable fit for a range of patient sizes. The sleeve is open at both ends to permit ease of placement and removal on the patient's arm or leg. The exterior surface of the sleeve has easily visible instructions to indicate that no blood pressure, I.V. or venipuncture is to be performed on the arm or leg which wears the sleeve.

### BACKGROUND OF THE INVENTION

1. Field of the Invention

The present invention is directed to a patient protection device and, more particularly, to a sleeve which is worn on a patient's arm or leg to alert attending medical personnel of the patient's potential risk for injury due to unwanted blood pressure measurements, intravenous access and venipuncture.

2. Discussion of the Related Art

Damage or removal of the lymph nodes can lead to chronic swelling of part of the body, usually an arm or a leg. This condition, known as lymphedema, is due to impaired flow of water and proteins through the lymph vessels. Primary lymphedema may be an inherited condition which is present at birth or, in some instances, may develop after puberty. Secondary lymphedema sometimes occurs after damage to or obstruction of the lymph nodes, as a result of surgery, infection or trauma. Patients who have had their lymph nodes removed as part of a surgical procedure, such as a mastectomy, are particularly at risk for the development of secondary lymphedema. When a patient's lymph nodes are removed, it is important to protect the affected arm or leg from trauma. In particular, the patient is advised to avoid blood pressure measurements, intravenous access and venipuncture from being performed on the affected arm or leg. It is important that patients who are at risk advise medical personnel of this potential injury prior to undergoing subsequent medical exams and procedures. In instances where the patient is hospitalized, it is common practice to place a sign above the patient's bed advising attending medical personnel to avoid blood pressure measurements, I.V.'s and venipuncture in the patient's affected arm or leg.

Unfortunately, nurses and other medical staff attending to the patient do not always remember to avoid the patient's affected arm or leg. When the attending medical personnel enters the patient's room during evening hours, they may not see any posted signs with instructions to avoid the affected limb. It is unfortunate that a significant number of patients are permanently injured as a result of having blood pressure measurements, I.V.'s and/or venipunctures performed on their affected arms or legs, despite having given attending medical personnel prior instructions. The potential for injury, particularly when a patient is sleeping, causes a great deal of discomfort and anxiety for the patient who is susceptible to onset of lymphedema.

### SUMMARY OF THE INVENTION

The present invention is directed to a safety device which is especially designed for patients who have had their lymph nodes removed or who otherwise require protection of an arm or leg during a medical examination, medical procedure or hospital stay. The safety device of the present invention is directed to a sleeve formed of a latex free fabric which is soft and comfortable for prolonged contact with the skin and which is adapted to stretch in order to accommodate various patient sizes. The sleeve is worn on a patient's affected arm or leg to protect the affected limb from any injury that may occur in a hospital or outpatient setting. The sleeve provides protection from unwanted blood pressure measurements, intravenous access, and venipuncture (e.g. blood draws). The exposed surface of the sleeve is provided with highly visible indicia which instructs attending medical personnel to avoid blood pressure measurements, I.V.'s and venipuncture on the affected arm or leg. The unmistakable presence of the sleeve on the patient' affected limb immediately alerts all attending medical personnel of the patient's potential risk for injury.

### BRIEF DESCRIPTION OF THE DRAWINGS

For a fuller understanding of the nature of the present invention, reference should be made to the following detailed description taken in conjunction with the accompanying drawings in which:

FIG. 1 is a top perspective view of the patient protection device of the present invention; and

FIG. 2 is a perspective view showing the patient protection device fitted on a patient's affected arm.

Like reference numerals refer to like parts throughout the several views of the drawings.

### DETAILED DESCRIPTION OF THE PREFERRED EMBODIMENT

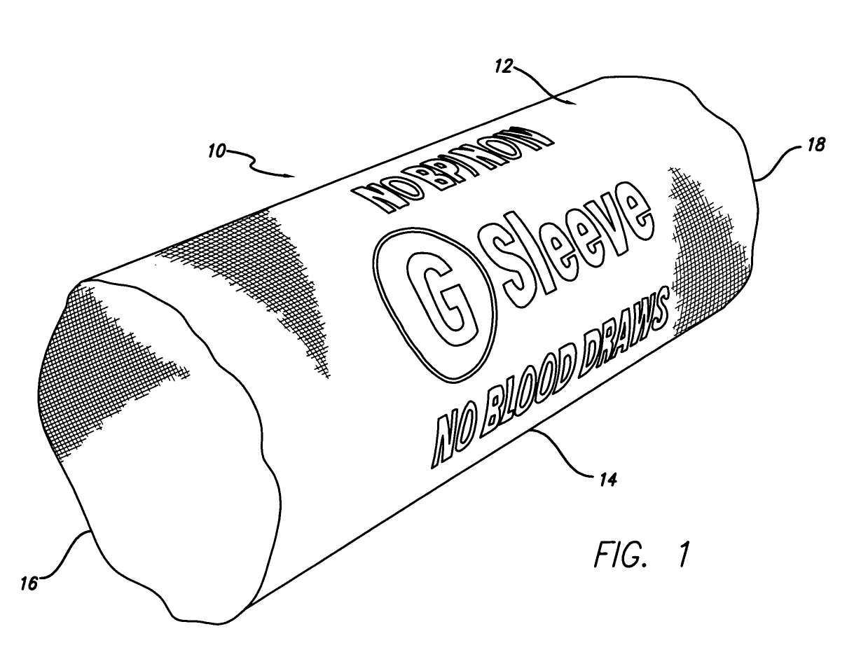

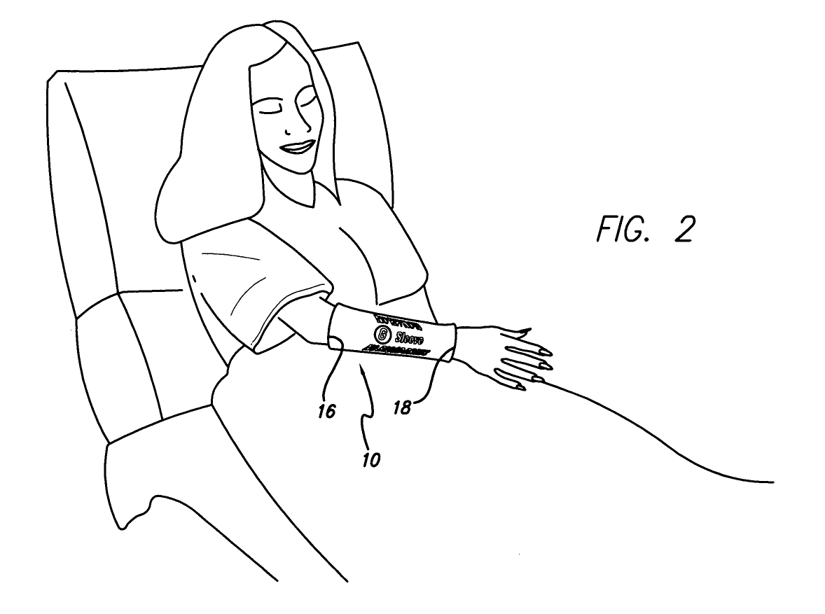

Referring to FIGS. 1 and 2 of the drawings, the patient protection device of the present invention is shown and generally indicated as 10. The patient protection device 10 is defined by a sleeve 12 having an elongate fabric body 14 which is open at opposite ends 16, 18 and formed in a generally tubular structure for surrounding a patient's arm or leg. The sleeve body 14 is formed entirely of a latex free, soft fabric which is stretchable and resilient to accommodate various size patients. In a preferred embodiment, the sleeve body fabric is cotton polyester. The exterior surface of the sleeve body is provided with printed indicia, such as by silkscreen, providing instructions to attending medical personnel to avoid blood pressure measurements, intravenous access and venipuncture (e.g., blood draws) on the arm or leg which wears the sleeve. In a preferred embodiment, the printed indicia provides the repeated instructions as follows:

NO BP/IV

NO BLOOD DRAWS

As shown in FIG. 2, the patient's arm or leg is passed through the open ends 16, 18 of the sleeve to position the sleeve on the affected arm or leg. The sleeve is left visibly exposed, preferably with no covering garments so that attending medical personnel are instantly alerted to avoid blood pressures, intravenous access, and blood draws or other venipuncture on the affected arm or leg. The patient protection device, when used as described above, prevents nursing errors and simplifies documentation for the attending medical personnel.

While the present invention has been shown and described in accordance with a preferred and practical embodiment thereof, it is recognized that departures from the instant disclosure are contemplated within the spirit and scope of the present invention.

What is claimed is:

1. A method for alerting medial personnel to the existence of a patient's condition and potential risk of injury to the patient, said method comprising the steps of:
providing a sleeve formed of a stretchable, resilient material and including a main tubular body with an outer surface, an inner surface and opposite open ends;
providing precautionary instructions on the exterior surface of the main tubular body of the sleeve to protect the patient from injury; and
fitting the sleeve onto a limb of the patient so that the sleeve and the precautionary instructions are visible to the medical personnel attending to the patient.

2. The method as recited in claim 1 wherein said precautionary instructions includes an instruction to avoid taking blood pressure measurements on the limb that is fitted with the sleeve.

3. The method as recited in claim 1 wherein said precautionary instructions include an instruction to avoid intravenous access on the limb that is fitted with the sleeve.

4. The method as recited in claim 1 wherein said precautionary instructions include an instruction to avoid taking blood draws from the limb that is fitted with the sleeve.

5. The method as recited in claim 1 wherein the precautionary instructions are intended to protect the patient from lymphedema.

6. The method as recited in claim 1 wherein said stretchable resilient material of said sleeve is a woven fabric.

7. A method for alerting medial personnel to the existence of a patient's condition and potential risk of injury to the patient, said method comprising the steps of:
providing a sleeve formed of a stretchable, resilient material and including a main tubular body with an outer surface, an inner surface and opposite open ends;
providing precautionary instructions on the exterior surface of the main tubular body of the sleeve alerting medical personnel to avoid the performance of procedures on a limb of the patient that is susceptible to injury; and
fitting the sleeve onto the limb of the patient that is susceptible to injury so that the sleeve and the precautionary instructions are visible to the medical personnel attending to the patient.

8. The method as recited in claim 7 wherein said precautionary instructions include an instruction to avoid taking blood pressure measurements on the limb that is fitted with the sleeve.

9. The method as recited in claim 7 wherein said precautionary instructions include an instruction to avoid intravenous access on the limb that is fitted with the sleeve.

10. The method as recited in claim 7 wherein said precautionary instructions include an instruction to avoid taking blood draws from the limb that is fitted with the sleeve.

11. The method as recited in claim 7 wherein the precautionary instructions are intended to protect the patient from lymphedema.

12. The method as recited in claim 7 wherein said stretchable resilient material of said sleeve is a woven fabric.
